(19) United States
(12) Patent Application Publication
(10) Pub. No.: US 2006/0257346 A1
(43) Pub. Date: Nov. 16, 2006

(54) Cosmetic composition for diminishing the appearance of lines and pores

(76) Inventors: Fatemeh Mohammadi, Hauppauge, NY (US); Lavinia C. Popescu, Jackson Heights, NY (US)

Correspondence Address: THE ESTEE LAUDER COS, INC, 125 PINELAWN ROAD, MELVILLE, NY, 11747, US

(21) Appl. No.: 11/128,027

(22) Filed: May 12, 2005

(51) Int. Cl. A61K 8/89

(52) U.S. Cl. 424/70.12; 424/401

(57) ABSTRACT

A cosmetic composition for reducing the appearance of lines, wrinkles and the size of pores in the skin is provided. The composition comprises a silica component, a silicon resin-coated elastomeric core component, and a cosmetic patch component, comprising algin/serine/atelocollagen, in a cosmetically acceptable aqueous vehicle.

### BACKGROUND OF THE INVENTION

The invention relates to a cosmetic composition for topical application to the skin. The invention more particularly relates to a composition that camouflages flaws in the skin to provide the skin with a smoother, more youthful appearance. Moreover, the compositions of the invention, on application to the skin, provide immediate reduction in the appearance of lines and wrinkles and the size of pores in the skin.

As skin ages, particularly the skin on the face, it begins to exhibit fine lines and then wrinkles. Additionally, whether skin is young or old, it can exhibit enlarged pores, whether from a blemish condition or simply because as the skin ages, it becomes less resilient, and the pores, which have become enlarged through stretching of the skin, remain so. It is desirable that the skin appear to retain its youthful, lineless and poreless appearance.

Typical formulations for reducing the appearance of lines and wrinkles in older skins tend to be comprised of different components from those components typically found in formulations used for reducing the appearance of pore size. The latter compositions, which generally are intended for younger, oilier skins, are usually combined with drying and/or oil absorbing components, and, therefore, are not suitable for older skins exhibiting lines and wrinkles.

The present invention provides a cosmetic composition, which when applied to the skin, provides instant reduction in the appearance of fine lines, wrinkles, particularly in the eye and lip areas, and the size of pores, to create the appearance of a smoother, more flawless and younger looking complexion. The compositions of the invention can be applied alone, under makeup or over makeup, to provide the appearance of smoother, more youthful, beautiful skin.

### SUMMARY OF THE INVENTION

The present invention relates to cosmetic compositions which, when applied to the skin, provide immediate reduction in the appearance of lines, wrinkles and pore size in the skin, while imparting to the skin a smoother, more youthful appearance. The compositions of the invention provide a unique combination of a silica component, a silicon resin-coated elastomeric core component and a cosmetic patch component comprising algin/serine/atelocollagen in a cosmetically and/or dermatologically acceptable aqueous vehicle.

The invention also relates to a method for reducing the appearance of lines, wrinkles and the size of pores in the skin, by applying to the skin exhibiting the lines, wrinkles and/or enlarged pores, a cosmetically or dermatologically effective amount of a composition comprising a silica component, a silicon resin-coated elastomeric core component, and a cosmetic patch component comprising, algin/serine/atelocollagen, in a cosmetically acceptable aqueous vehicle.

### BRIEF DESCRIPTION OF THE DRAWING

FIG. 1 is a chart illustrating the percent reduction in the appearance of pores and lines and wrinkles immediately after the application to skin of a composition of the present invention.

### DETAILED DESCRIPTION OF THE INVENTION

The novel cosmetic compositions of the invention are aqueous formulations containing a silica component, a silicon resin-coated elastomeric core component and a cosmetic patch component comprising algin/serine/atelocollagen. The composition, when applied to the skin, particularly the facial skin, provides an immediate reduction in the appearance of fine lines, wrinkles and/or the size of the pores. The compositions of the invention are substantially oil-free formulations. By “substantially oil-free”, it is intended that, if oil is present in the composition, the amount of oil is no more than 5 weight percent.

The first component of the compositions of the invention is silica or a coated or encapsulated silica which imparts mattifying benefits to the novel formulation. The silica component possesses a reflectance property which, when the composition is applied to the skin, reduces the appearance of lines, wrinkles, and the size of pores in the skin. The silica may be any type of silica suitable for cosmetic use, such as silica provided in bead form. Examples of silicas useful in carrying out the invention are silica microspheres, such as MSS-500/3; Spheron P-1500; Cab-o-sil M7-D; silica beads SB-700; Monoveil and Monoveil Spectra T. Monoveil and Monoveil Spectra T, which are available from Ried International Corporation (Brentwood, N.Y.), as a result of their multi-layered and spherical forms, refract visible light rays inside the sphere's layers, reflecting the light at various angles. The light scattering effect provided blurs fine lines, wrinkles, and pores while also providing transparency. Coated or encapsulated silicas useful in the compositions of the present invention, include, for example, silica/triethoxycaprylsilane, silica/iron oxide/triethoxycaprylsilane, silica/methicone, silica/dimethicone, silica/titanium dioxide/dimethicone, silica/titanium dioxide/tin oxide, silica/iron oxides, silica/iron oxides/kaolin/PVP/water, silica/isononyl isonanoate, silica/octyidodecyl stearoyl stearate/isononyl isononanoate, silica//methoxy amodimethicone silsesquioxane copolymer, silica/ultramarines/kaolin/PVP/water, silica silylate, and hexyldecyldiisocyanate/trimethylol hexylacetone crosspolymer//silica. The preferred silica components for use in the present invention are Monoveil and Monoveil Spectra T.

The first component is present in the compositions of the invention in a range in the amount of about 5 to about 40 weight percent, preferably in the range of from about 10 to about 30 weight percent, and more preferably in the range of from abut 10 to about 20 weight percent, based on the total weight of the composition.

The second component of the compositions of the invention is a silicon resin-coated elastomeric core material. The silicon resin provides a soft, cushioning feel, and the elastomeric core provides slip lubricity. Together, these materials provide the compositions of the invention with properties which impart a unique silky texture to the skin to which the composition is applied. Silicon resin-coated elastomeric core components suitable for use in the invention formulations are any which are cosmetically and dermatologically acceptable, and preferably are synthetic silicone- or siloxane-containing copolymers, such as dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, organopolysiloxanes, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, water/propylene glycol/cyclomethicone D5/polysilicone-11/butylenes glycol/glycerine/isohexadecane/ammonium polyacryloyidimethyl, butylenes glycol/water/dimethicone/polysilicone-11/glycerin/isohexadecane/ammonium polyacryloyidimethyl taurate/retinyl palmitate, dimethicone/polysilicone-11/cyclomethicone cyclomethicone/trimethylsiloxysilicate, and spherical powders comprising silicone elastomer coated with silicone resin. Preferably, the copolymer used in the compositions of the subject invention is a hybrid silicone powder whose structure consists of silicone rubber coated with silicone resin. Particularly preferred is vinyl dimethicone/methicone silsesquioxane crosspolymer, such as KSP-100, available from Shin Etsu Chemical Company., Ltd. (Tokyo, Japan).

The second component is present in the composition in the range of about 5 percent to about 40 weight percent, preferably in the range of about 10 to about 30 weight percent, and more preferably, in the range of about 10 to about 20 weight percent, based on the total weight of the composition.

As the third component of the invention compositions, use is made of a material which will provide the unique compositions of the present invention with the double benefit of enhancing the adherence of the powder components to the skin while minimizing water loss from cutaneous tissue. This cosmetic patch component includes algin/serine/atelocollagen. Particularly preferred for use in the compositions of the invention is the Moisturizing Marine MICROPATCH®, available from Coletica (Northport, N.Y.). The collagen and the alginate are capable of associating with the skin; forming a filmogenic micronetwork, which acts to prevent the evaporation of moisture from the skin, and which holds serine. The serine is slowly released from the network into the skin for extended moisturization. The amino acid, serine, is a synthesis precursor of ceramides, which are naturally occurring skin lipids. It is believed that the release of serine into the upper layers of the epidermis stimulates the production of ceramides in the skin.

The third component is present in the composition in the range of about 0.01 to about 20 weight percent, preferably in the range of about 0.1 to about 10 weight percent, and more preferably, in the range of about 1 to about 5 weight percent, based on the total weight of the composition.

The compositions of the invention include any base that is aqueous or that is miscible in water. The medium is therefore predominantly water but it can also include solvents such as, for example, hydroalcohol, glycerin, and combinations thereof. Examples of acceptable hydroalcohols include, but are not limited to, ethanol, propanol or glycols such as butylene glycol. It is preferred that the polyol be a C2-C5 alcohol.

The compositions of the subject invention also may include skin conditioning agents, such as hydrogenated lecithin, polypropylene terephthalate, butylene glycol, and polyglycerin modified silicones, for example, polyglyceryl-3 disiloxane dimethicone, for example, KF-6100 (Shin-Etsu, Tokyo, Japan). The silicones also demonstrate a light scattering property and can contribute to achieving a soft focus effect on the skin. The compositions of the invention may also include, for example, thickeners; gelling agents, such as acrylates/C10-30 alkyl acrylate crosspolymer, carbomer, and xanthan gum. The compositions of the present invention may further comprise one or more useful cosmetically and dermatologically acceptable plant extracts and biological actives, which include, but are not limited to anti-acne agents, such as, for example, Laminaria saccharina extract, 10-hydroxydecanoic acid, and phytosphingosine; whitening agents, such as for example, licorice root extract, ferulic acid and hinokito; firming ingredients, such as, for example, vitamin C and/or derivatives thereof, such as aminopropyl ascorbyl phosphate, glycerylpolymethacrylate/PEG-8/palmitoyloligopeptide and acetylhexapeptide-3 (argireline); and lifting agents, such as, for example, sweet almond seed extract, pea extract and algae extract/pullulan.

Compositions of the invention may be utilized in various topical forms, including emulsions, such as creams, lotions, serums and cream gels, and anhydrous formulations, including ointments and sticks.

The following non-limiting examples further illustrate the embodiments of the invention.

### EXAMPLES

### Example 1

### Composition of the Invention—Lotion

TABLE 1

| | WEIGHT |
|---|---|
| MATERIALS | PERCENT |
| sequence 1 | |
| purified water | 52.25 |
| ammonium acrylodimethyltaurate/VP copolymer | 0.70 |
| sequence 2 | |
| disodium EDTA | 0.05 |
| sequence 3 | |
| butylene glycol | 0.50 |
| polyglyceryl-3 disiloxane dimethicone | 1.00 |
| vinyl dimethicone/methicone silsesquioxane crosspolymer | 10.00 |
| hydrogenated lecithin | 1.50 |
| hexyldecyldiisocyanate/trimethylol hexylacetone | 10.00 |
| crosspolymer//silica | |
| dimethicone crosspolymer-3/isododecane | 2.00 |
| denatured alcohol | 20.00 |
| purified water/butylene glycol/algin/atelocollagen/serine | 2.00 |
| TOTAL | 100.00 |

The sequence 1 ingredients are combined and gently mixed for about 20 minutes at room temperature until the mixture forms a clear gel. The temperature of the mixture is gradually increased until a temperature of 70° C. is reached at which time the sequence 2 component is introduced to the mixture. Sequence 3 ingredients are mixed together separately, and the temperature of the mixture is gradually increased to 75° C. The mixture of sequence 3 ingredients is introduced to the mixture with stirring. The combined mixture is gradually cooled to 35° C. and mixed until homogenized.

### Example 2

The composition of Example 1 is tested on sixteen women (panelists) having large pores and having lines in the eye area. The product is applied one time on the day of the test. A measured amount (approximately 0.5 cc) is dispensed from a syringe to the hands of the subject by the investigator. The subject then is instructed to apply the product to the skin on the face with the fingertips.

Optical Reduction in the Appearance of Lines and Wrinkles

Prior to, and immediately after product application, the appearance of lines and wrinkles on each panelist's skin is assessed and documented with close-up photography. Photographs of the face are taken with a Nikon M3 digital camera. Photographs are evaluated using an image analysis program, Optimas 6.5. Lines and wrinkles are assessed by examining changes in the Integrated Optical Density (IOD) before and after product application. A decrease in IOD represents a decrease in fine lines and wrinkles.

Standard Procedure for using the Optimas 6.5 Program:

Instrumentation:

1. Fuji S2 or Nikon M3 digital camera

2. Computer station with Optimas 6.5 (Media Cybernetics, San Diego, Calif.) Image Analysis Program

Photography:

1. Panelists heads are placed in a head rest to insure reproducibility of positioning.

2. The camera is positioned one foot from the panelist at an F stop of 32. An unpolarized photograph of the area demonstrating fine lines and/or wrinkles is taken.

Program Operation:

1. The photographs are opened and displayed in the Optimas 6.5 program.

2. Using editing tools in the Optimas 6.5 software, a region of interest (ROI) is masked on the area where lines are visible. When evaluating the lines before and after product use, the same ROI is used at each time point.

3. The program automatically segments the darker, indented areas (i.e. lines).

4. Each photograph is analyzed by the computer program for the area occupied by the lines in the segmented ROI, and a grey scale value is assigned to the area. A lower area represents the presence of fewer lines, while a higher area represents the presence of more lines. A lower grey value represents the presence of deeper lines, while a higher grey value represents the presence of less deep lines. The grey scale is shown below.

Grey Scale:

The grey scale is widely used by photographers and the scientific community as a method to assign a number value to the “lightness” or “darkness” of an image. The scale starts at zero (black), and ends at 255 (white). Everything between 0 and 255 is a shade of grey. A picture of the scale is shown below:

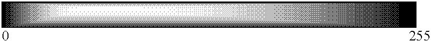

When wrinkles are photographed, a grey value is assigned to the “darkness” of each wrinkle. A deeper wrinkle appears darker than a shallow wrinkle; thus, a deeper wrinkle is assigned a lower grey value.

5. Lines and wrinkles are assessed by examining changes in the Integrated Optical Density (IOD) before and after product use. IOD is equal to [(255-grey value) x area]. A decrease in IOD represents a decrease in the number and/or the depth of fine lines and wrinkles, while an increase in IOD represents an increase in the number and/or the depth of fine lines and wrinkles.

6. The image analysis program is interfaced with Microsoft Excel and automatically places the values in an Excel spreadsheet.

Optical Reduction in the Appearance of Pore Size

The appearance of each panelist's pores is assessed prior to, and immediately after product application using close-up photography. The panelist's head is place in a head rest to insure reproducibility of positioning. The camera is positioned at a distance of 2 feet from the panelist with an F stop of 32. Photographs of the right and the left sides of the panelist's face are taken using a Nikon M3 digital camera. Grading is in accordance with a 10 point analog scale, and a percent change from baseline is calculated. Clinical evaluations of pore visibility are conducted by an investigator who has an extensive perceptual vocabulary, and who is experienced in scale usage and the use of standardized evaluation techniques. A standard lexicon and references for that specific parameter (i.e. a photo scale depicting the typical appearance of a “0”, of a “2”, etc., up to “10”) are used for evaluation.

10 point scale:

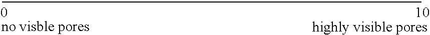

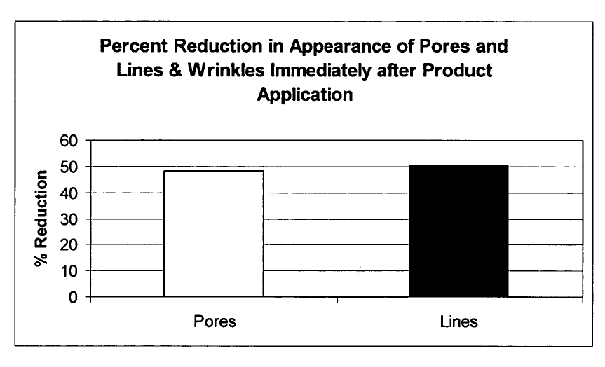

As shown in FIG. 1, the panelists demonstrated, on average, a 48% reduction in the appearance of pores and a 51% reduction in the appearance of lines and wrinkles immediately after the application of the product.

Numerous variations and modifications of the invention will become readily apparent to those familiar with cosmetic products. Accordingly, the scope of the invention should not be construed as limited to the specific examples described, as those examples are presented herein only as being illustrative of the many formulations possible according to the invention.

What is claimed is:

1. A topical cosmetic and/or dermatological composition for reducing the appearance of fine lines, wrinkles and the size of pores in the skin, the composition comprising a silica component, a silicon resin-coated elastomeric component, and a cosmetic patch component comprising, algin/serine/atelocollagen, in a cosmetically acceptable aqueous vehicle.

2. The composition according to claim 1, in which the silica component is silica or an encapsulated or coated silica.

3. The composition according to claim 2, in which the silica component is selected from the group consisting of silica/triethoxycaprylsilane, silica/iron oxide/triethoxycaprylsilane, silica/methicone, silica/dimethicone, silica/titanium dioxide/dimethicone, silica/titanium dioxide/tin oxide, silica/iron oxides, silica/iron oxides/kaolin/PVP/water, silica/isononyl isonanoate, silica/octyldodecyl stearoyl stearate/isononyl isononanoate, silica//methoxy amodimethicone silsesquioxane copolymer, silica/ultramarines/kaolin/PVP/water, silica silylate, and hexyldecyldiisocyanate/trimethylol hexylacetone crosspolymer//silica.

4. The composition according to claim 2, wherein the silica component is silica.

5. The composition according to claim 4, wherein the silica component is a multi-layered silica.

6. The composition according to claim 1, in which the silicone resin-coated elastomeric core component is selected from the group consisting of dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, organopolysiloxanes, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, water/propylene glycol/cyclomethicone D5/polysilicone-11/butylenes glycol/glycerine/isohexadecane/ammonium polyacryloyldimethyl, butylenes glycol/water/dimethicone/polysilicone-11/glycerin/isohexadecane/ammonium polyacryloyldimethyl taurate/retinyl palmitate, dimethicone/polysilicone-11/cyclomethicone cyclomethicone/trimethylsiloxysilicate, and vinyldimethicone/methicone silsesquioxane crosspolymer.

7. The composition according to claim 6, in which the silicone resin-coated elastomeric core component is vinyldimethicone/methicone silsesquioxane crosspolymer.

8. The composition according to claim 1, which comprises in the range of from about 5 to about 40 weight percent of the silica component, in the range of from about 5 to about 40 weight percent of the silicon resin-coated elastomeric core component, and in the range of from about 0.01 to about 20 weight percent of the cosmetic patch component.

9. The composition according to claim 8, which comprises in the range of from about 10 to about 30 weight percent of the silica component, in the range of from about 10 to about 30 weight percent of the silicon resin-coated elastomeric core component, and in the range of from about 0.1 to about 10 weight percent of the cosmetic patch component.

10. The composition according to claim 9, which comprises in the range of from about 10 to about 20 weight percent of the silica component, in the range of from about 10 to about 20 weight percent of the silicon resin-coated elastomeric core component, and in the range of from about 1 to about 5 weight percent of the cosmetic patch component.

11. A method for reducing the appearance of one or more of fine lines, wrinkles and the size of pores in the skin, the method comprising applying to the skin exhibiting the lines, wrinkles and/or pores, a cosmetically or dermatologically effective amount of a composition comprising a silica component, a silicon resin-coated elastomeric core component, and a cosmetic patch component comprising, algin/serine/atelocollagen, in a cosmetically acceptable aqueous vehicle.

12. The method according to claim 11, in which the silica component of the composition is silica or an encapsulated or coated silica.

13. The method according to claim 12, in which the encapsulated or coated silica is selected from the group consisting of silica/triethoxycaprylsilane, silica/iron oxide/triethoxycaprylsilane, silica/methicone, silica/dimethicone, silica/titanium dioxide/dimethicone, silica/titanium dioxide/tin oxide, silica/iron oxides, silica/iron oxides/kaolin/PVP/water, silica/isononyl isonanoate, silica/octyldodecyl stearoyl stearate/isononyl isononanoate, silica//methoxy amodimethicone silsesquioxane copolymer, silica/ultramarines/kaolin/PVP/water, silica silylate, and hexyldecyldiisocyanate/trimethylol hexylacetone crosspolymer//silica.

14. The method according to claim 12, wherein the silica component of the composition is a multi-layered silica.

15. The method according to claim 11, in which the silicon resin-coated elastomeric core component of the composition is selected from the group consisting of dimethicone//dimethicone/vinyl dimethicone crosspolymer, phenyl trimethicone//dimethicone/phenyl vinyl dimethicone crosspolymer, cyclopentasiloxane//dimethicone/vinyl dimethicone crosspolymer, dimethicone/polysilicone-11, organopolysiloxanes, cyclopentasiloxane/polysilicone-11, phenyl trimethicone/polysilicone-11, cyclomethicone/polysilicone-11/petrolatum, cyclomethicone/polysilicone-11, stearoxy methicone/dimethicone copolymer, water/propylene glycol/cyclomethicone D5/polysilicone-11/butylenes glycol/glycerine/isohexadecane/ammonium polyacryloyldimethyl, butylenes glycol/water/dimethicone/polysilicone-11/glycerin/isohexadecane/ammonium polyacryloyldimethyl taurate/retinyl palmitate, dimethicone/polysilicone-11/cyclomethicone cyclomethicone/trimethylsiloxysilicate, and vinyldimethicone/methicone silsesquioxane crosspolymer.

16. The method according to claim 15, in which the silicon resin-coated elastomeric core component is vinyldimethicone/methicone silsesquioxane crosspolymer.

17. The method according to claim 11, in which the composition comprises in the range of from about 5 to about 40 weight percent of the silica r component, in the range of from about 5 to about 40 weight percent of the silicon resin-coated elastomeric core component, and in the range of from about 0.01 to about 20 weight percent of the cosmetic patch component.

18. The method according to claim 18, in which the composition comprises in the range of from about 10 to about 30 weight percent of the silica component, in the range of from about 10 to about 30 weight percent of the silicon resin-coated elastomeric core component, and in the range of from about 0.1 to about 10 weight percent of the cosmetic patch component.

19. The method according to claim 19, in which the composition comprises in the range of from about 10 to about 20 weight percent of the silica component, in the range of from about 10 to about 20 weight percent of the silicon resin-coated elastomeric core component, and in the range of from about 1 to about 5 weight percent of the cosmetic patch component.
